(19) United States
(12) Patent Application Publication
(10) Pub. No.: US 2025/0334162 A1
(43) Pub. Date: Oct. 30, 2025

(54) COMPLIANT SUPPORT MECHANISM

(71) Applicants: Jonah LEINWAND, Waterloo (CA); Stewart MCLACHLIN, Waterloo (CA)

(72) Inventors: Jonah LEINWAND, Waterloo (CA); Stewart MCLACHLIN, Waterloo (CA)

(21) Appl. No.: 18/605,753

(22) Filed: Mar. 14, 2024

Related U.S. Application Data
(60) Provisional application No. 63/452,081, filed on Mar. 14, 2023.
(60) Provisional application No. 63/540,771, filed on Sep. 27, 2023.

(51) Int. Cl. F16F 1/02; A61F 2/44; B33Y 10/00

(52) U.S. Cl. CPC F16F 1/025; B33Y 10/00; A61F 2/442

(57) ABSTRACT

There is disclosed a compliant support mechanism including a first support surface; a second support surface; and one or more flexure prongs arranged in a configuration therebetween, each flexure prong having a plurality of bends or curves along its length so as to allow the configuration of one or more flexure prongs to be resiliently compliant when subjected to external forces on the support surfaces. The one or more flexure prongs have a compliance profile dependent upon the application.

### CROSS-REFERENCE TO RELATED APPLICATIONS

This application claims the benefit of U.S. Provisional Application No. 63/452,081, filed on Mar. 14, 2023, and entitled COMPLIANT SUPPORT MECHANISM, and the benefit of U.S. Provisional Application No. 63/540,771, filed on Sep. 27, 2023, and entitled COMPLIANT SUPPORT MECHANISM, the entirety of which are incorporated herein by reference.

### FIELD

The present disclosure is related to compliant support mechanisms, and more generally to mechanisms, systems and methods for providing structural support, damping, and a controlled range of motion in a wide range of applications.

### BACKGROUND

Compliant support mechanisms can be found in various different fields, including but not limited to: mechanical systems, robotics, aerospace, optics, implants, prosthetics, and medical devices. For example, in the medical devices space, the field of spinal surgery has seen significant advancements in recent years with the development of minimally invasive surgical techniques and the introduction of new types of replacement implants. However, existing replacement designs may suffer from complications such as implant migration, subsidence, wear, and other forms of failure.

What is needed are improved mechanism designs and processes which address at least some of the limitations of prior art compliant support mechanisms.

### SUMMARY

The present disclosure is related to compliant support mechanisms, and more specifically to mechanisms, systems and methods for providing structural support, damping, and/or a controlled range of motion in a wide range of applications. These applications include, but are not limited to, mechanical systems, robotics, aerospace, optics, implants, prosthetics, and medical devices.

In an aspect, the compliant support mechanism comprises a novel design aimed at providing improved performance and greater customizability of a desired compliance or flexure profile, whether in terms of the amount of support, damping, flexibility, stiffness, and/or a controlled range of motion.

In an embodiment, additive manufacturing techniques are used to produce a monolithic compliance support mechanism having an upper supporting surface and a lower supporting surface. A plurality of compliant flexure prongs are arranged in a desired configuration therebetween, with each end of the compliant flexure prongs monolithically joined with the upper support surface and the lower support surface at either end.

In another embodiment, the compliant flexure prongs have a variable structure including one or more bends, curves, or twists, such that the compliant flexure prong is able to provide support, damping, and/or a controlled range of motion based on the shape, structure, and material profile of the compliant flexure prongs used.

In another embodiment, the placement, position and orientation of the compliant flexure prongs on each of the upper support surface and the lower support surface is determined by a desired direction or orientation of damping or a desired direction or orientation of a controlled range of motion.

In another aspect, the present disclosure is related to artificial disc replacement (e.g. compliant flexures), more specifically pertaining to systems and methods for replacing damaged or diseased intervertebral discs with implantable devices that mimic the natural motion and function of healthy discs in the spine. The present disclosure provides a novel design for an artificial disc, aimed at providing improved outcomes for patients suffering from a wide range of spinal conditions, including but not limited to degenerative disc disease, herniated discs, and other forms of damaged or diseased intervertebral discs.

In another embodiment, additive manufacturing techniques may be used to quickly and efficiently build a customized compliant support mechanism which is designed to precisely fit a desired application, such as for example a replacement intervertebral disc for a specific patient based on measurements taken from the patient.

In another embodiment, pseudo-customized implants may be used where the implants or components of the implant may be pre-made, but finely adjustable and/or customizable by the surgeon before implanting via surgery. This may involve adjustment of parts, assembly of different components, testing of fabricated designs or swapping of materials to custom fit the implants after measurements are taken from a patient.

In another embodiment, computational modeling and machine learning tools are employed to design, track, analyze, and optimize the performance of implant designs over the long term. Based on the long term performance of particular implant designs, various features of those longer lasting implants may be adopted into other implant designs to achieve improved durability and performance over the long term.

In another embodiment, computational modeling and machine learning tools are employed to assist with designing a custom implant based on a particular compliance or flexure profile for a given patient. Based on particular measurements and characteristics of the given patient, the custom implant is configured to provide optimal performance and durability. An ecosystem of various custom implant design parameters could be generated using different numerical methods or computational modeling techniques to examine how different design parameters, such as flexure geometry, flexure layout, and flexure materials affect the resulting compliance profile.

Thus, the inventors seek to address challenges in the prior art by providing a novel artificial disc replacement system that offers improved motion and function, while also minimizing the risk of wear. Common artificial disc designs typically consist of multi-part devices that interface at a ball and socket joint. However these devices suffer drawbacks such as wear of the surfaces, and mechanical properties different from a natural intervertebral disc, which can lead to further complications and adjacent segment failure in the spine. The presented system includes a range of implantable devices with shared key characteristics that can be used to replace damaged or diseased intervertebral discs, and can be customized to meet more specific uses.

In an embodiment, the presented system and process includes a range of implantable devices with shared key characteristics that can be used to replace damaged or diseased intervertebral discs, and can be customized to meet more specific uses.

In another embodiment, the upper and lower support surfaces or plates can vary in shape, thickness, length, width, porosity, curvature, and features which are sturdy and provide fixation to the vertebrae.

In another embodiment, the support surfaces could be linked to adjacent compliant supports on either end to create the stacked concepts. The interface to link the surfaces could be achieved through additive manufacturing or bonding, for example. The support surfaces could also be offset so that they are not perfectly coincident or concentric.

In some embodiments, the upper and lower support surfaces may be porous or include a lattice/infill or additional features for alignment or fixation of the structure such as one or more keels, and a relative roughness for accommodating bonding at will.

In another embodiment, there are at least two flexure prongs present in the body of the device that cross the midline of the center of the body in opposite directions, where the flexure prongs can vary in thickness, width, and shape of cross-sectional area. The flexure prongs are attached to the endplates in a smooth manner where the endplates and flexure prongs are made of the same material, such that the device as a whole is a compliant mechanism with no articulating joints. Alternatively, the flexure prongs could be made of multiple materials or fibers stacked or laminated together to provide a desired compliance profile.

In another embodiment, the shape of the flexure prongs is described by some non-linear, piecewise function where the flexure prongs have some number of bends in them, which may be multi-directional bends, non-linear bend paths could include reversing directions, and producing greater than one region of compliance which allow the flexure prongs and as a whole to flex, and extend or contract. In preferred (created) embodiments of the device, there are 2 or 3 flexure prongs that each consist of 3 bends, with at least 1 of the flexure prongs in each case going in one direction across the cross-sectional midline, and at least 1 other flexure prong going in the opposite direction.

### BRIEF DESCRIPTION OF THE DRAWINGS

The present invention, and the objects of the invention will become apparent when consideration is given to the following detailed description thereof. Such description makes reference to the annexed drawings, wherein:

FIG. 1 shows an illustrative compliant support mechanism for use in an intervertebral replacement disc implant in accordance with an embodiment;

FIGS. 2A-2C show illustrative examples of compliant support mechanisms for use in an intervertebral replacement disc implant in accordance with additional embodiments;

FIGS. 3A-3C illustrate how changing the position of the flexure prong attachment points on the upper and lower support surfaces change the structures of the compliant support mechanisms and their respective support profiles.

FIG. 4A shows a detailed view of an illustrative expanded join between a flexure prong and a support surface to provide additional strength.

FIG. 4B shows an illustrative example of a compliant support mechanism using such expanded joins.

FIG. 5 shows an illustrative example of a flexure prong having a multi-stage flexure prong profile.

FIGS. 6A-6D show different views of a support surface in accordance with an illustrative embodiment.

FIGS. 7A-7C show additional illustrative examples of support surfaces having features for securing an end of a separate flexure prong on the support surface in an assembly concept.

FIGS. 8A and 8B show different views of a support surface having recessed pockets or indentations for receiving an end of a flexure prong at specific locations on the support surface.

FIGS. 9A and 9B show examples of two or three flexure prongs connecting upper and lower support surfaces.

FIGS. 9C and 9D show examples of how the location of the ends of the flexure prongs on the upper and lower support surfaces may be varied.

FIGS. 10A and 10B show illustrative examples of flexure prongs with complex profiles, including multiple bends possibly within multiple axis, and including bends which may extend beyond the edge of the upper and lower support surfaces.

FIGS. 11A-11C show different views of a multi flexure prong design having bends in multiple dimensions at once.

FIGS. 12A-12C show different views of a multi flexure prong design having different thicknesses and widths in various curved shapes in accordance with an illustrative embodiment.

FIGS. 13A-13D show various examples of two and three flexure prong designs having multiple curves and flexure points, and different arrangements in terms of connections to the upper and lower support surfaces to achieve different compliance profiles.

FIG. 14A-14C show examples of multi-stage compliant support mechanisms in which flexure prongs are arranged between a lower support surface, one or more middle support surfaces, and an upper support surface.

FIGS. 15A-15C show additional examples of multi-stage compliant support mechanisms with different types of flexure prongs and arrangements between multiple support surfaces.

FIGS. 16A-16C show an illustrative example of a multi-stage compliant support mechanism in which some flexure prongs may pass through an aperture of a middle support layer and connect between upper and lower support surfaces.

FIGS. 17A-17E show illustrative examples of flexure prongs including a plurality of cuts, notches or pores along the flexure path to allow an additional degree of compliance in comparison to a design where the plurality of notches are not provided.

FIGS. 18A and 18B show an illustrative example of a compliance support mechanism in which only one flexure prong connects one support stage to another, and the layering arrangement provides a greater degree of compliance in comparison to multiple flexure prong designs.

FIGS. 19A-19D show various examples of flexure prongs having different cross-sections and locations.

FIG. 20A shows examples of flexure prongs, each having a varying cross section and meandering curves over its length.

FIG. 20B shows additional examples of flexure prongs having multiple variations in the number of bends or curves.

FIG. 20C shows an illustrative example of a flexure prong which splits into two or more branches.

FIG. 21A shows an illustrative example of a lattice arrangement on a support surface for facilitating a tighter bond between the support surface and additional material which will be placed within the gap between the plates.

FIG. 21B shows an illustrative example of a compliant support mechanism having rough, porous or uneven surfaces provided on the support layer to provide better coupling with, or alignment to, adjacent surfaces.

FIG. 22A shows an illustrative example of the lattice arrangement of FIG. 21A provided on a 3D printed example of a compliant support mechanism.

FIG. 22B shows an illustrative example of a compliant support mechanism having an unobstructed open center area shown in dashed lines, between support layers.

FIG. 23A shows an illustrative example of a support surface having an elongate groove for guiding or constraining a bearing in accordance with an embodiment.

FIG. 23B shows a close up of the elongate groove of FIG. 23A.

FIG. 24A shows an illustrative example of a support surface having an oval groove for guiding or constraining a bearing in accordance with an embodiment.

FIG. 24B shows a close up of the oval groove of FIG. 24A.

FIG. 25A shows an illustrative example of a support surface having a raised oval fence for guiding or constraining a bearing in accordance with an embodiment.

FIG. 25B shows a close up of the oval fence of FIG. 25A.

FIG. 26A shows an egg shaped concave depression for constraining the egg shaped bearing in accordance with an embodiment.

FIG. 26B shows a close up view of the egg shaped concave depression.

FIG. 27A shows an egg shaped bearing being constrained within a correspondingly shaped concave depression in accordance with an embodiment.

FIG. 27B shows a spherical bearing being constrained within a correspondingly shaped concave depression in accordance with an embodiment.

FIG. 28A shows a spherical bearing being constrained between an upper support surface and a lower support surface in accordance with an embodiment.

FIG. 28B shows another view of a spherical bearing being constrained between an upper support surface and a lower support surface in accordance with an embodiment.

FIG. 29 is a schematic block diagram of a computer device which may provide a suitable operating environment for one or more embodiments.

### DETAILED DESCRIPTION

As noted above, the present disclosure is related to compliant support mechanisms, and more specifically to systems and methods for providing structural support, damping, and/or a controlled range of motion in a wide range of applications. These applications include, but are not limited to, mechanical systems, robotics, aerospace, optics, implants, prosthetics, and medical devices.

In an aspect, the compliant support mechanism comprises a novel design aimed at providing improved performance and greater customizability of a desired compliance profile, whether in terms of the amount of support, damping, and/or a controlled range of motion.

In an embodiment, additive manufacturing techniques are used to produce a monolithic compliance support mechanism having an upper supporting surface and a lower supporting surface. A plurality of compliant flexure prongs are arranged in a desired configuration therebetween, with each end of the compliant flexure prongs monolithically joined with the upper support surface and the lower support surface at either end.

In another embodiment, the compliant flexure prongs have a variable structure including one or more bends, curves, or twists, such that the compliant flexure prong is able to provide support, damping, and/or a controlled range of motion based on the shape and structure profile of the compliant flexure prongs used.

In another embodiment, the placement and position of the compliant flexure prongs on each of the upper support surface and the lower support surface is determined by a desired direction or orientation of damping or a desired direction or orientation of a controlled range of motion.

In another aspect, the present disclosure is related to artificial disc replacement (e.g. compliant flexures), more specifically pertaining to systems and methods for replacing damaged or diseased intervertebral discs with implantable devices that mimic the natural motion and function of healthy discs in the spine. The present disclosure provides a novel design for an artificial disc, aimed at providing improved outcomes for patients suffering from a wide range of spinal conditions, including but not limited to degenerative disc disease, herniated discs, and other forms of damaged or diseased intervertebral discs.

In another embodiment, additive manufacturing techniques may be used to quickly and efficiently build a customized compliant support mechanism which is designed to precisely fit a desired application, such as for example a replacement intervertebral disc for a specific patient based on measurements taken from the patient.

In another embodiment, pseudo-customized implants may be used where the implants may be pre-made, but finely adjustable and customizable before implanting via surgery.

In another embodiment, computational modeling and machine learning tools are employed to track and analyze the performance of implants over the long term. Based on the long term performance of particular implant designs, various features of those longer lasting implants may be adopted into other implant designs to achieve improved durability and performance over the long term.

The inventors seek to address these challenges by providing a novel artificial disc replacement system that offers improved motion and function, while also minimizing the risk of wear. Common artificial disc designs typically consist of multi-part devices that interface at a ball and socket joint. However these devices suffer drawbacks such as wear of the surfaces, and mechanical properties different from a natural intervertebral disc, which can lead to further complications and adjacent segment failure in the spine. The presented system includes a range of implantable devices with shared key characteristics that can be used to replace damaged or diseased intervertebral discs, and can be customized to meet more specific uses.

In another aspect, the present disclosure describes a novel artificial disc replacement system that offers improved motion and function, while also minimizing the risk of wear. Common artificial disc designs typically consist of multi-part devices that interface at a ball and socket joint. However these devices suffer drawbacks such as wear of the surfaces, and mechanical properties vastly different from a natural intervertebral disc, which can lead to further complications and adjacent segment failure in the spine.

In an embodiment, the presented system and process includes a range of implantable devices with shared key characteristics that can be used to replace damaged or diseased intervertebral discs, and can be customized to meet more specific uses.

In another embodiment, the upper and lower support surfaces or plates can vary in thickness, length, and width, which are sturdy and provide fixation to the vertebrae.

In some embodiments, the upper and lower support surfaces may include a lattice/infill/porosity, and a relative roughness for accommodating bonding at will. In another embodiment, there are at least two flexure prongs are present in the body of the device that cross the midline of the center of the body in opposite directions, where the flexure prongs can vary in thickness, width, and shape of cross-sectional area. The flexure prongs are attached to the endplates in a smooth manner where the endplates and flexure prongs are made of the same material, such that the device as a whole is a compliant mechanism with no articulating joints.

In another embodiment, the shape of the flexure prongs is described by some non-linear, piecewise function where the flexure prongs have some number of bends in them producing greater than one region of compliance which allow the flexure prongs and as a whole to flex and extend. In preferred (created) embodiments of the device, there are 2 or 3 flexure prongs that each consist of 3 bends, with at least 1 of the flexure prongs in each case going in one direction across the cross-sectional midline, and at least 1 other flexure prong going in the opposite direction.

Various embodiments will now be described with reference to the drawings.

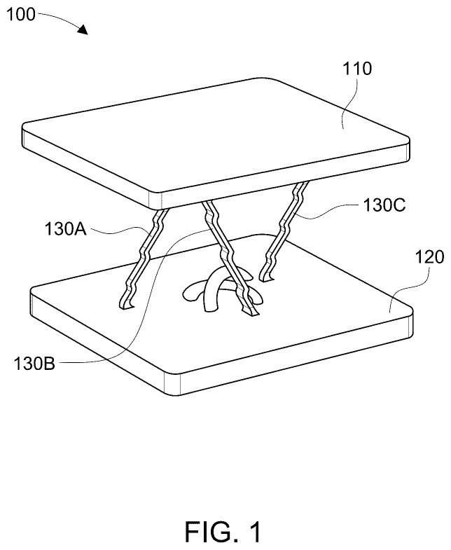

Referring to FIG. 1, shown is an illustrative compliant support mechanism 100 for use in an intervertebral replacement disc implant in accordance with an embodiment. As shown, the compliant support mechanism comprises a plurality of compliant flexure elements 130A, 130B, 130C connecting top 110 and bottom 120 supporting surfaces.

As will be explained in more detail below, the plurality of compliant flexure elements or prongs 130A, 130B, 130C may be configured in a variety of configurations, provided that each compliant flexure element includes at least one or more bends or curves which allow each compliant flexure element to resiliently stretch or shorten based on external forces subjected to the compliant support mechanism.

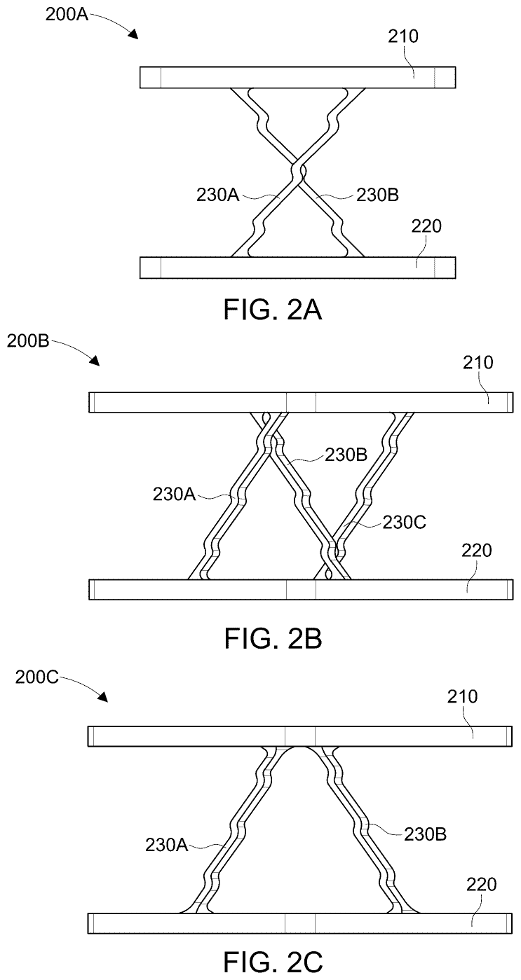

FIGS. 2A-2C show additional illustrative examples of compliant support mechanisms 200A, 200B, 200C for use in an intervertebral replacement disc implant. For example, the support surfaces or endplates 210, 220 can be the same, or they may be different. The gap in between each support surface or endplate 210, 220 may also vary, depending on the particular compliant flexure prongs 230A, 230B, 230C separating the support surfaces 210, 220.

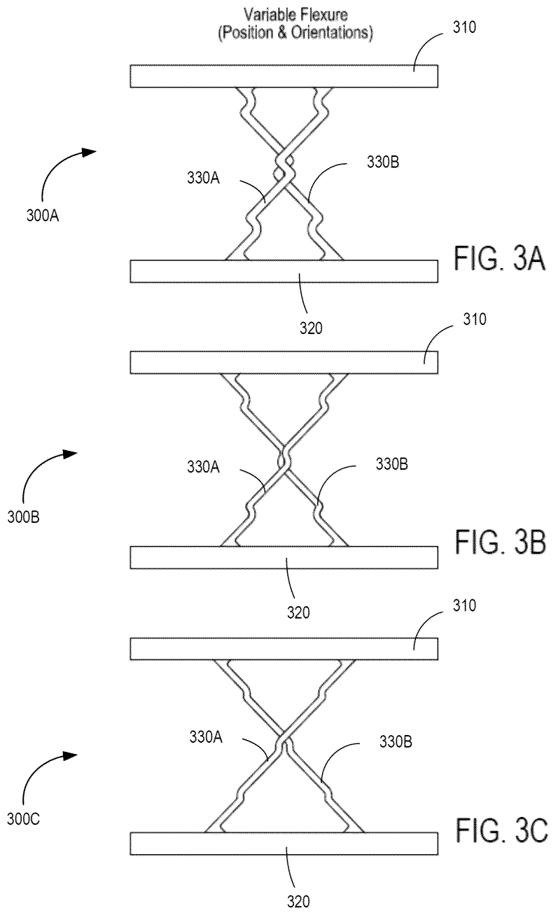

FIGS. 3A-3C illustrate compliant support mechanisms 300A, 300B, 300C showing how changing the position of the flexure prong 330A, 330B attachment points on the upper 310 and lower 320 support surfaces change the structures of the compliant support mechanisms and their respective support profiles. Thus, a particular arrangement or placement of the flexure prongs 330A, 330B may be altered to achieve varying gaps between endplates.

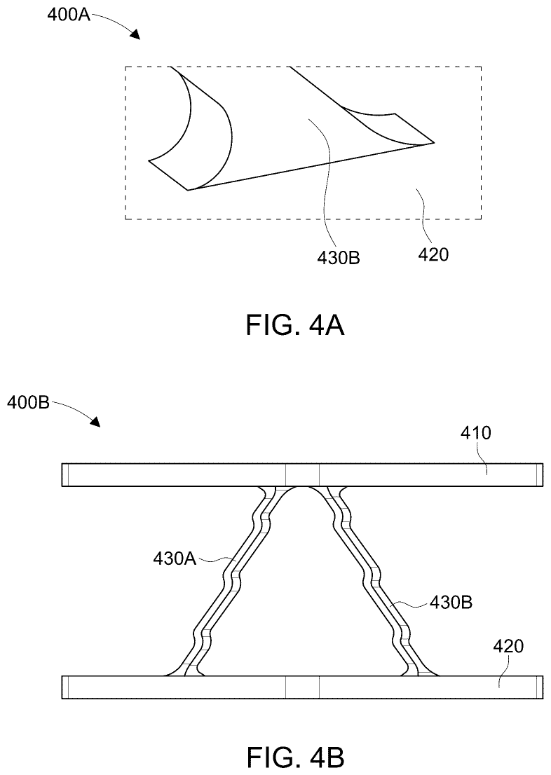

Now referring to FIG. 4A, shown is a detailed view 400A of an illustrative expanded join between a flexure prong 430B and a support surface 420 to provide additional strength. FIG. 4B shows an illustrative example of a compliant support mechanism 400B with top 410 and bottom 420 plates and flexure prongs 430A, 430B using such expanded joins for increased strength. As will be described in more detail below, this expanded join may be produced in an additive manufacturing process where the support surfaces, expanded join, and flexure prongs are all printed in a monolithic structure from the same or multiple 3D printer materials. By way of example and not by way of limitation, the type of 3D printing used may be LPBF (laser powder bed fusion).

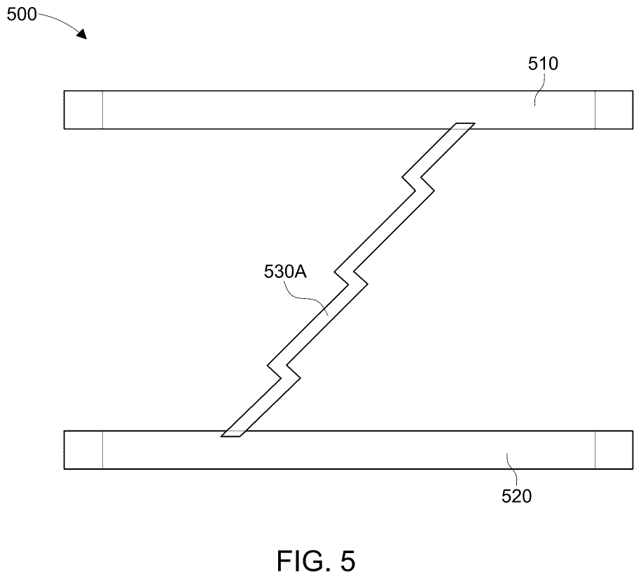

Now referring to FIG. 5, shown is an illustrative example of a flexure prong 530A having a multi-stage flexure prong profile. Gaps between multiple support plates 510, 520 can have different measurements based on the particular selection arrangement of a suitable flexure prong 530A, and a desired compliance or flexural profile achieved by combing multiple levels of compliant support mechanisms 500.

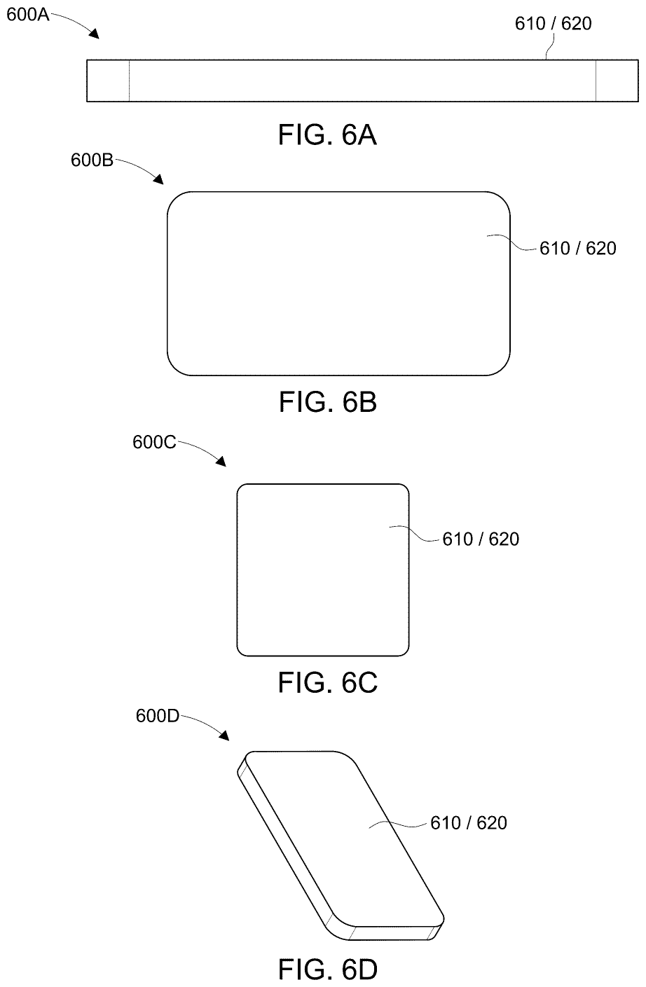

Now referring to FIGS. 6A-6D shown are different views 600A, 600B, 600C, 600D of a support surface 610, 620 in accordance with an illustrative embodiment. This illustrative example shows the support surface 610, 620 being generally rectangular, but it could also be square, round, ovular etc., and each support surface 610, 620 in a compliant support mechanism may be of a different or irregular shape to be a custom fit for a particular application.

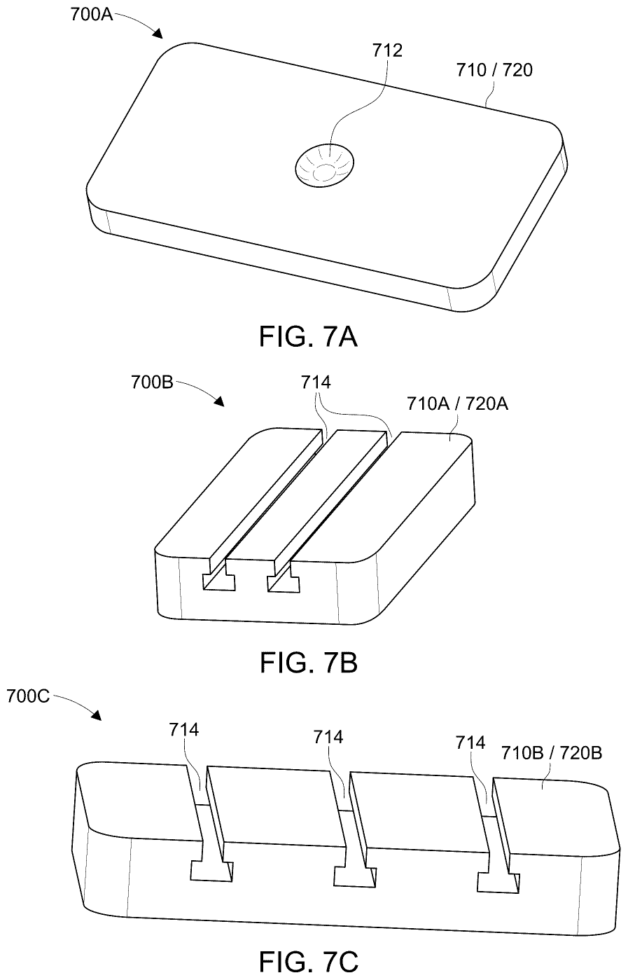

Now referring to FIGS. 7A-7C, shown are additional illustrative examples 700A, 700B, 700C of support surfaces 710, 720 having features for securing an end of a flexure prong on the support surface. For example, FIG. 7A shows a divot or bowl 712 which may receive a curved or round end of a flexure prong, which may then be secured in position via an adhesive, or via other securing means such as heating or laser welding. These features could also serve for alignment of the support surface in a particular position and/or orientation with respect to the mating bodies.

FIGS. 7B and 7C show slots 714 which have a widened base such that an enlarged end of a flexure prong can be inserted and its position adjusted along the length of the slot. This would allow the compliant support member to be adjusted such that it provides a custom height, and a custom compliance profile based on the configuration of the flexure prongs used.

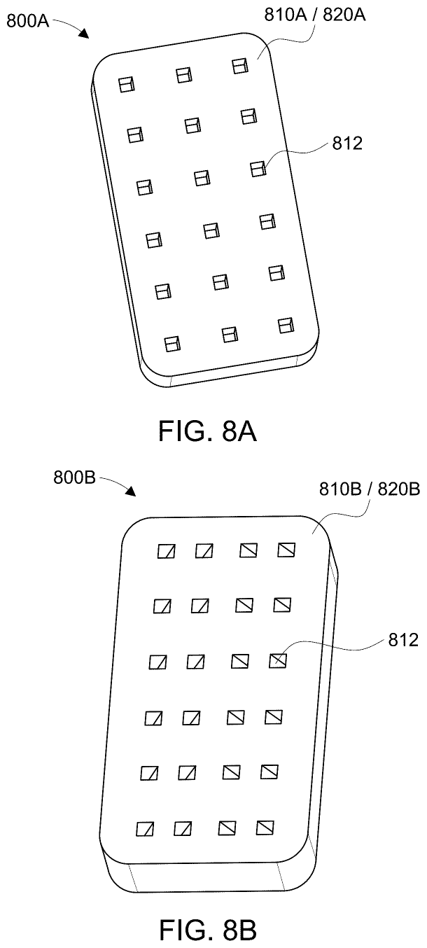

Now referring to FIGS. 8A and 8B show different views 800A, 800B of a support surface 810A, 810B and 820A, 820B having recessed pockets or indentations 812 for receiving an end of a flexure prong at specific locations on the support surface. By positioning a flexure prong in a different location, the distance between the support surfaces can be adjusted, as well as the compliance profile depending on whether the flexure prongs are positioned in an inner location, an outer location, evenly distributed, or focused in one or more areas to bias the manner in which the compliant support member will bend or flex based on an applied external force.

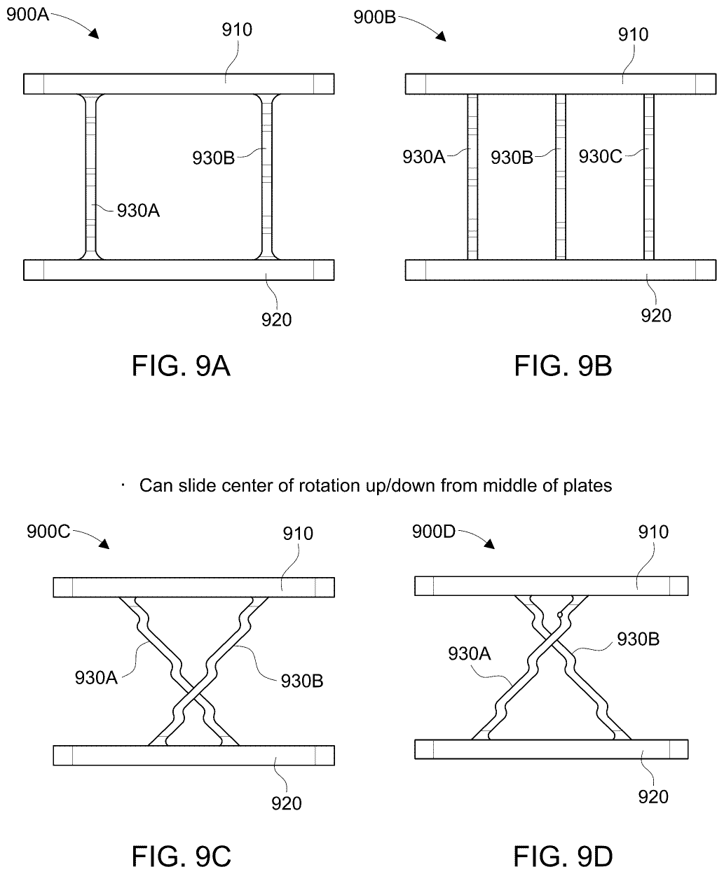

Now referring to FIGS. 9A and 9B, shown are examples 900A, 900B, 900C, 900D of two or three flexure prongs 930A, 930B, 930C connecting upper and lower support surfaces 910, 920. FIGS. 9C and 9D show examples of how the location of the ends of the flexure prongs on the upper and lower support surfaces may be varied by placement into different pockets or indentations of the support surfaces shown in FIGS. 8A and 8B, or slid along slots 714 shown in FIGS. 7B and 7C. For example, in FIG. 9C, the bottom support surface may have a tendency to be relatively less stable in comparison to the top support surface given the closure position of the two flexure prongs. In FIG. 9D, the situation would be reversed.

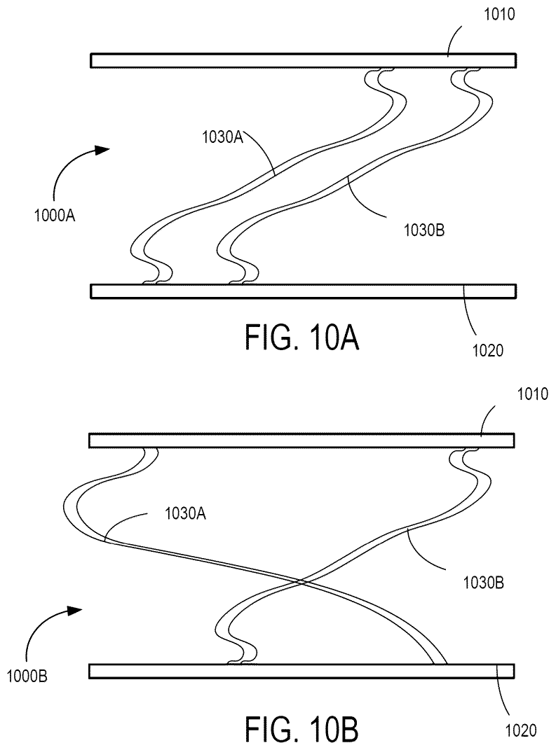

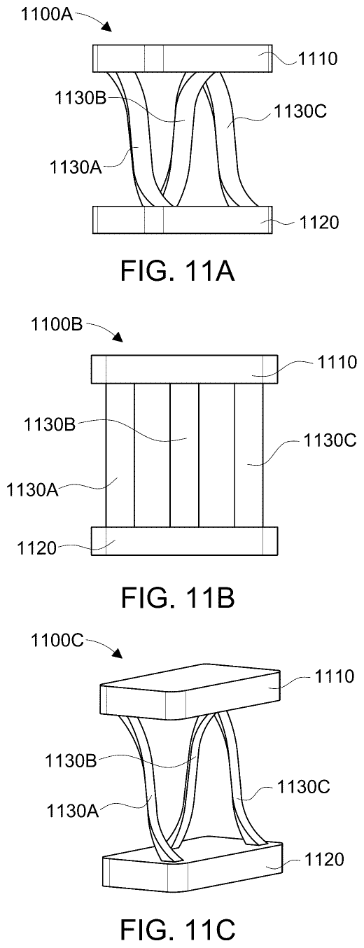

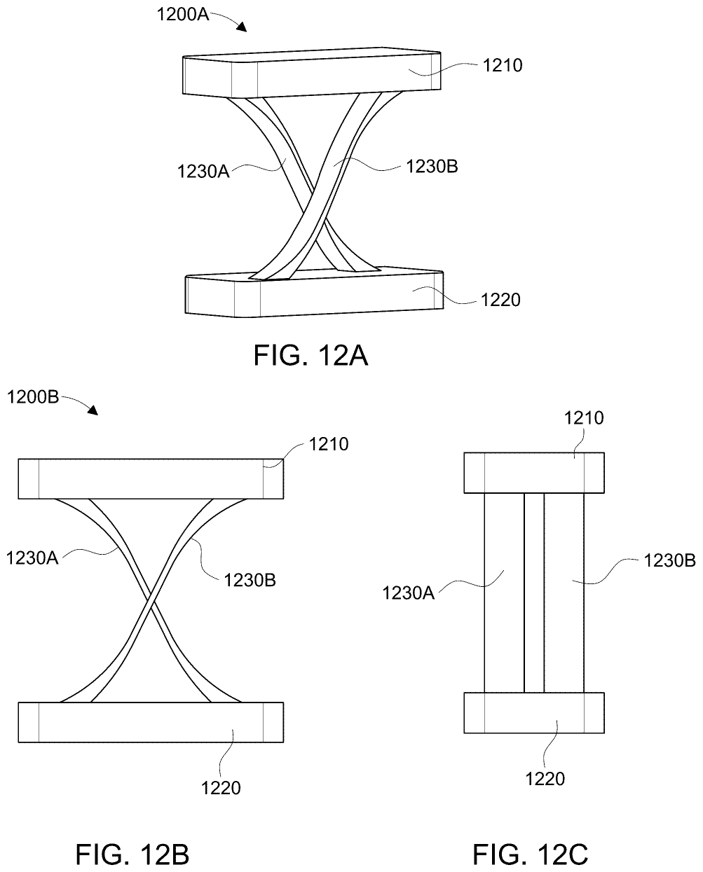

Now referring to FIGS. 10A and 10B, shown are illustrative examples of flexure prongs 1030A, 1030B with complex profiles, including multiple bends possibly within multiple axis, and including bends which may extend beyond the edge of the upper and lower support surfaces 1010, 1020. As shown, the meandering bends or curves can bend in any direction, progressing towards or away from the support surfaces, whether in a single plane, or in multiple dimensions at the same time. The meandering curves or bends can also have different profiles and locations along the length of the flexure prong, as shown by way of another example in FIGS. 11A-11C, and FIGS. 12A-12C.

FIGS. 11A-11C show compliant support mechanisms 1100A, 1100B, 1100C with upper and lower support surfaces 1110, 1120, and flexure prongs 1130A, 1130B, 1130C in various different arrangements.

FIGS. 12A-12C show compliant support mechanisms 1200A, 1200B, 1200C with upper and lower support surfaces 1210, 1220, and flexure prongs 1230A, 1230B in various different arrangements.

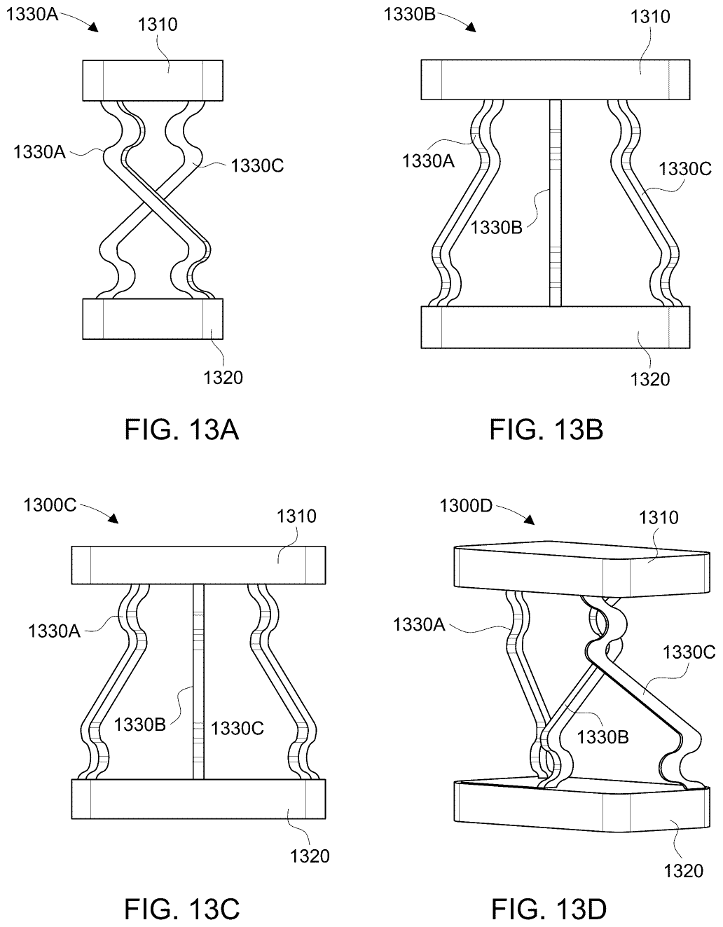

Now referring to FIGS. 13A-13D, shown are additional illustrative examples 1300A, 1300B, 1300C, 1300D of flexure prongs 1330A, 1330B, 1330C having multiple curves or bends that may be positioned in different arrangements and directions. Thus, one or more flexure elements can be connected in series or in parallel or crossing paths at varying angles between the supporting top and bottom surfaces 1310, 1320 to achieve a desired compliance or flexural profile for the structural support device, characterized by a range of motion in six degrees of freedom in Cartesian space (x, y, z) or about an arbitrary 3D joint axis.

In an embodiment, the device can be made of different materials including ferrous and non-ferrous metals, polymers, or other biomaterials.

For a preferred embodiment for use in orthopaedic implants, biocompatible materials such as titanium or stainless steel would be suitable.

The device can be manufactured using an additive manufacturing approach to enable a monolithic (single bodied and continuous) design. The device can also be manufactured from a collection of manufacturing techniques and assembled into the final structure.

When the device is manufactured from non-additive manufacturing approaches, the device can be assembled through slotting or inserting the flexure elements into slots or holes in the endplates (as shown above with reference to, where multiple slots exist in different positions along the endplate to allow for further customization of the compliance profile of the structural support device shown in FIGS. 7B and 7C, for example.

The device can also contain additional features on the inside or outside of the supporting endplates to provide adhesion and improve connection to other materials or surfaces on the top or bottom of the endplates, or within the internal cavity space.

In an embodiment, the internal cavity space is left open other than features added for improving connection and the flexure elements that pass through, such that an empty space exists that could be filled with a secondary material.

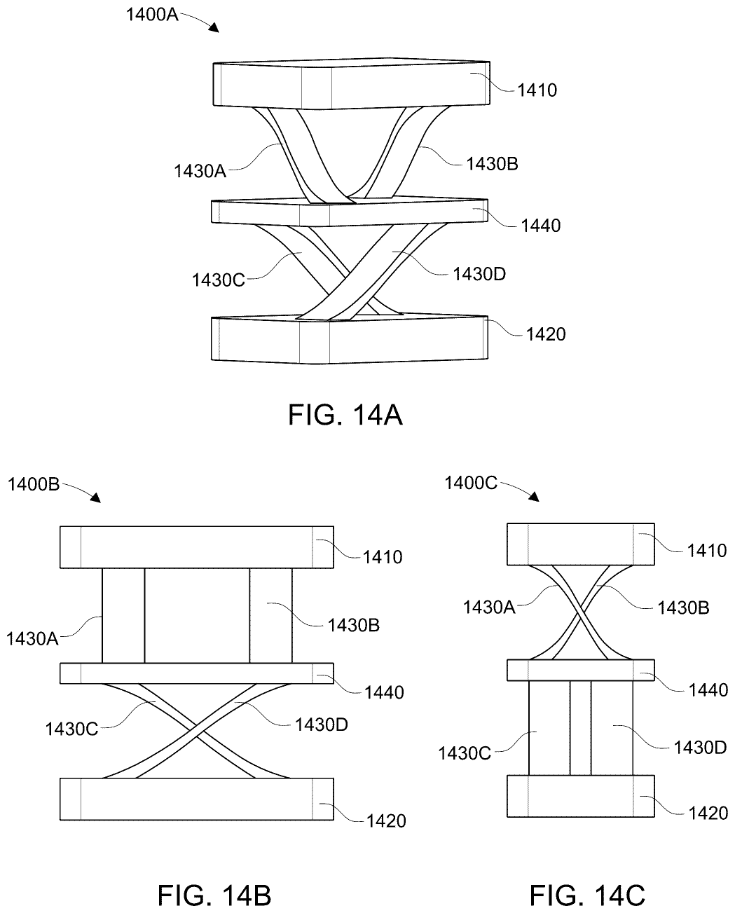

Now referring to FIGS. 14A-14C, shown are examples of multi-stage compliant support mechanisms 1400A, 1400B, 1400C in which flexure prongs 1430A, 1430B, 1430C, 1430D are arranged between a lower support surface 1420, one or more middle support surfaces 1440, and an upper support surface 1410. For example, this multi-stage concept would benefit needing to span a longer gap or distance with the compliant support. The multi-stage compliant supports having different flexure prong direction profiles could achieve a desired multiple degree of freedom compliance profile.

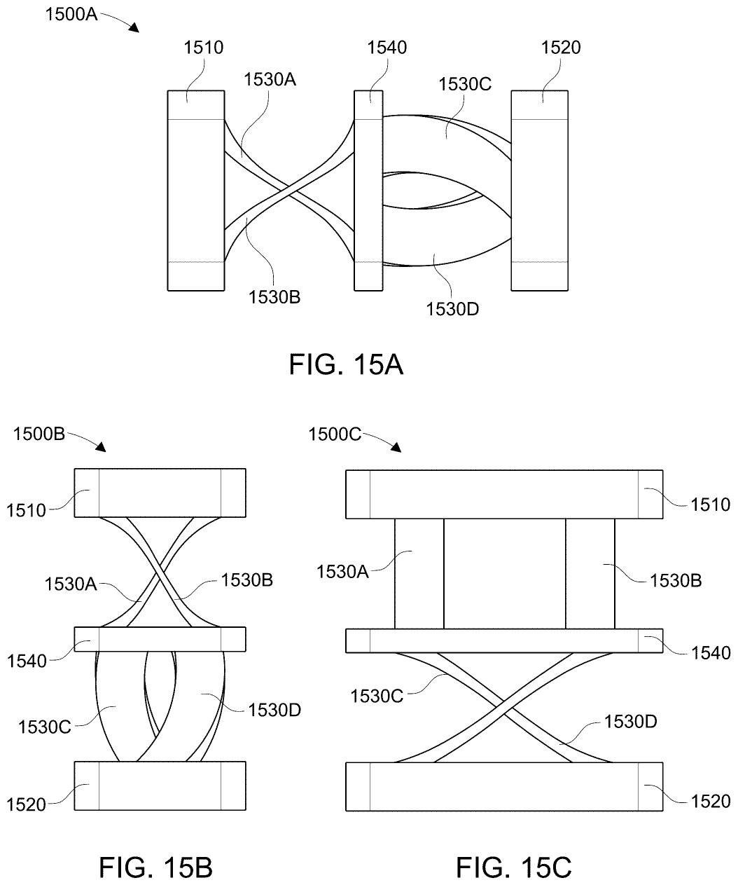

FIGS. 15A-15C also show various examples 1500A, 1500B, 1500C of two and three flexure prong 1530A, 1530B, 1530C, 1530D designs having multiple curves and flexure points, and different arrangements in terms of connections to the upper 1510, middle 1520, and lower 1540 support surfaces to achieve different compliance profiles. These additional examples of multi-stage compliant support mechanisms with different types of flexure prongs and arrangements between multiple support surfaces show a wide variety of shapes and configurations that may be used.

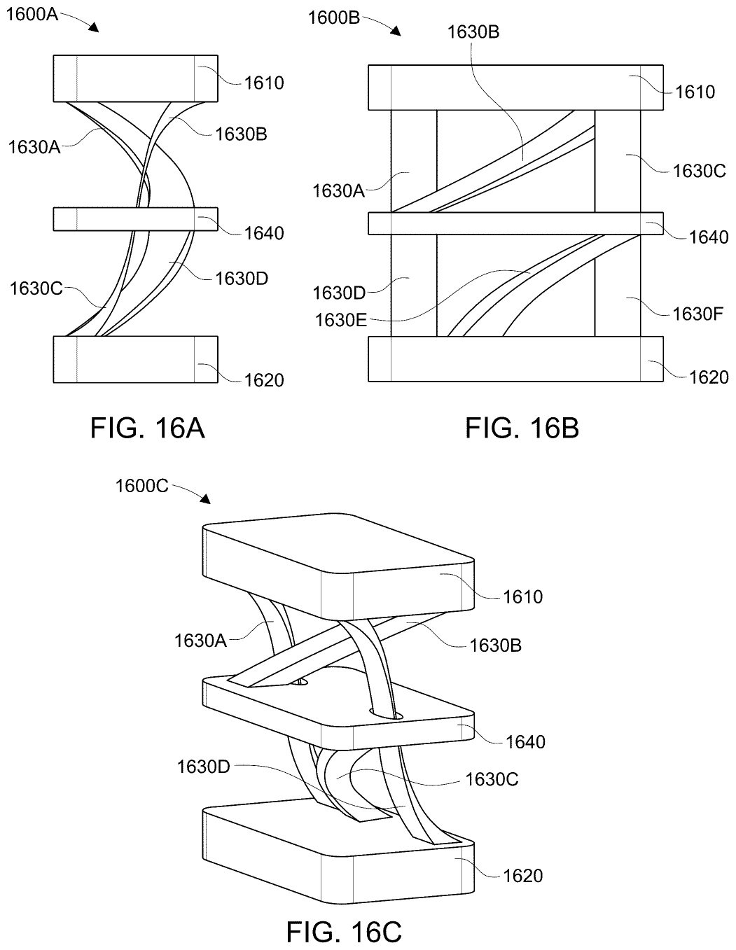

Now referring to FIGS. 16A-16C, show an illustrative examples of a multi-stage compliant support mechanisms 1600A, 1600B, 1600C in which some flexure prongs 1630A, 1630B, 1630C, 1630D may pass-through an aperture of a middle support layer 1640 and connect between upper 1610 and lower 1620 support surfaces. This allows for greater flexibility in compliance profiles by providing one or more pass-through apertures to connect the flexure prongs in different configurations.

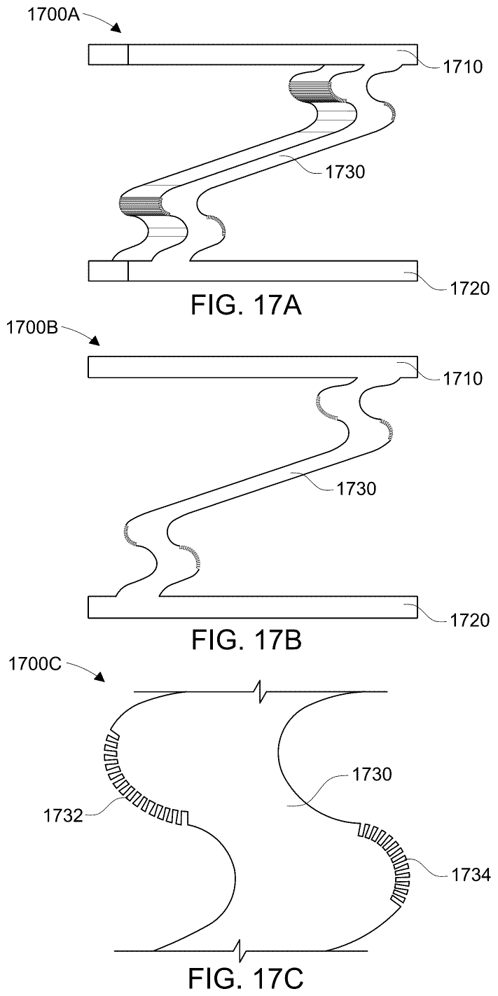

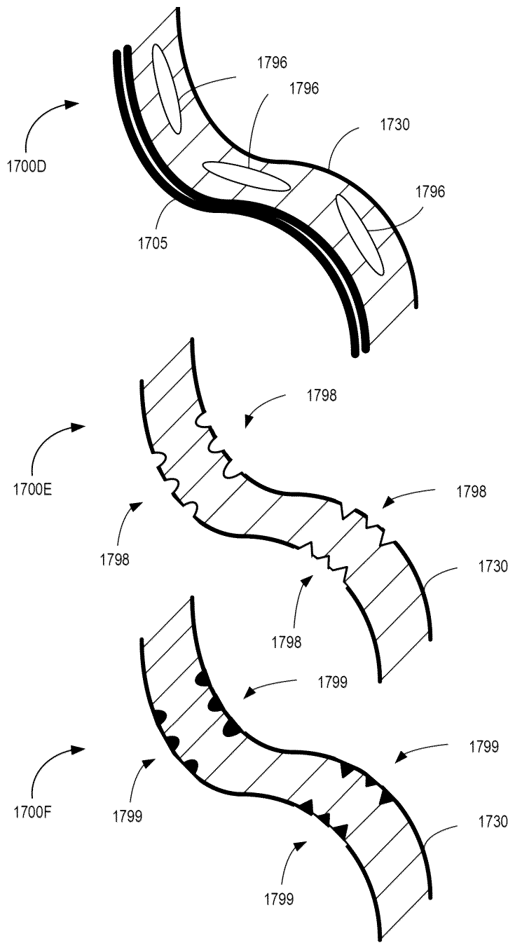

FIGS. 17A-17C show illustrative examples 1700A, 1700B, 1700C of flexure prongs 1730 including a plurality of notches, cuts or pores 1732, 1734 along the flexure path to allow an additional degree of flexibility or compliance in comparison to a design where the plurality of notches, cuts or pores are not provided.

FIG. 17D shows at 1700D a flexure 1730 with designed negative space or pores 1796 in the body of the flexure prong 1730. The image shows engineered defects or notches of the flexure prong edge, over varying shape and location, in order to change the compliance profile of the mechanism. FIG. 17D also shows that the flexure prong 1730 can be strengthened with a second material 1705, which may provide different flexure properties. The second material may also be a fibrous or laminate material to provide additional strength.

FIG. 17E shows at 1700E series of engineered defects or notches 1798 running only along one edge of the flexure prong 1730 of varying spacing and location, at locations including but not limited to, the bends and body of the flexures. A series of asymmetric engineered defects or notches are shown on both flexure sides. The bottom image shows a series of flexure engineered defects or notches all varying in size, position, and length.

FIG. 17F shows at 1700F that the series of engineered defects or notches 1798 can be filled with another type of material, in order to provide a different flexure profile than if the notches are not filled.

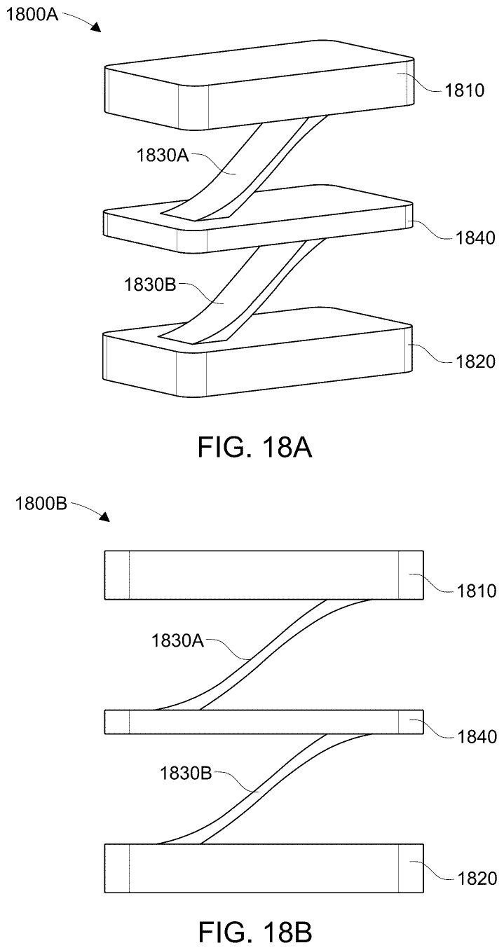

Now referring to FIGS. 18A and 18B, shown is an illustrative example of a compliance support mechanism 1800A, 1800B in which only one flexure prong 1830A, 1830B connects one support stage 1810, 1820 to another 1840, and the layering arrangement provides a greater degree of compliance in comparison to multiple flexure prong designs.

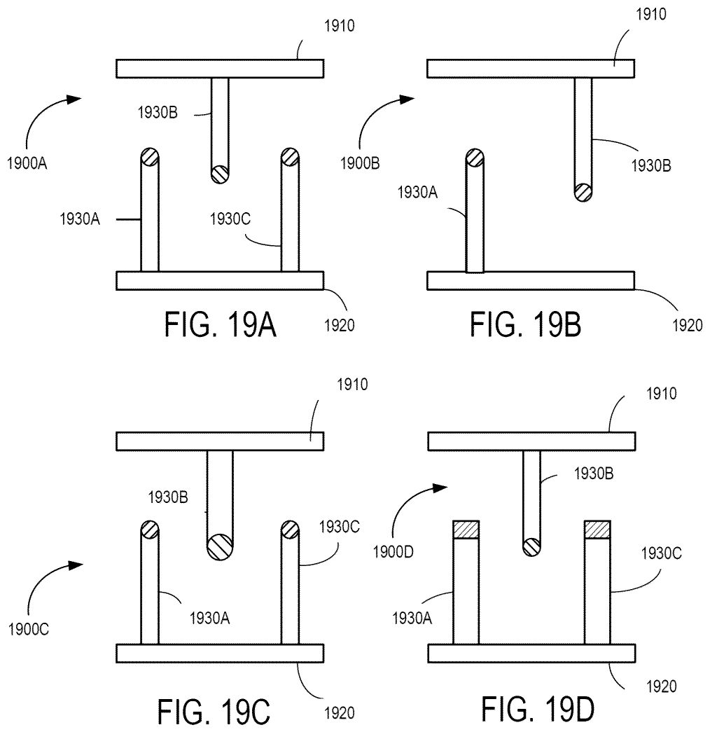

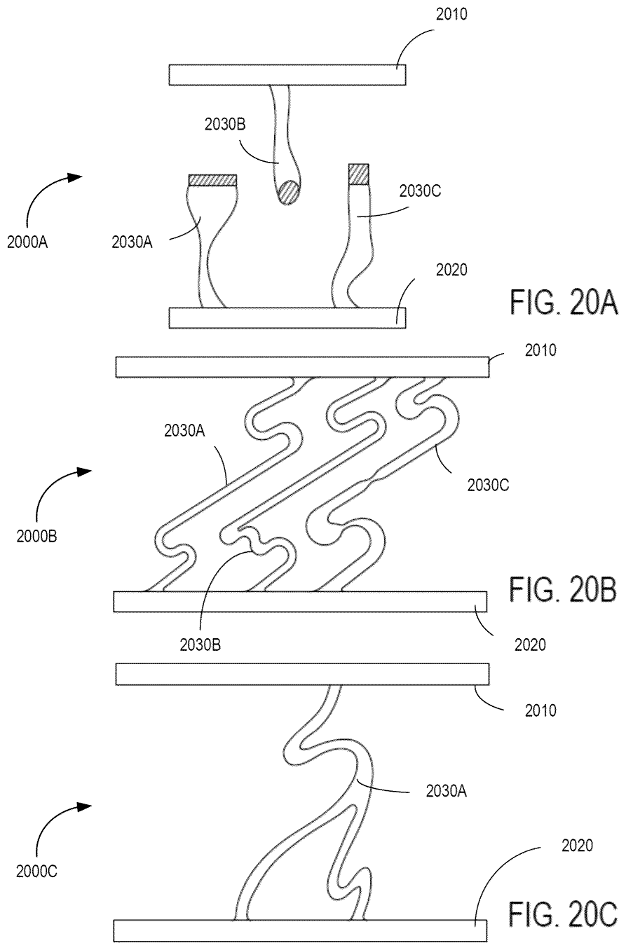

FIGS. 19A-19D show various examples 1900A, 1900B, 1900C, 1900D of flexure prongs 1930A, 1930B, 1930C having different cross-sections and locations attached to upper 1910 and lower 1920 support surfaces. For example, some flexure prongs may have a round profile of different sizes, a square or rectangular profile, or a non-standard shape such as a slanted oval or other profile.

Now referring to FIG. 20A, shown are examples 200A, 200B, 200C of additional flexure prongs 2030A, 2030B, 2030C, each having a varying cross section and meandering curves over its length between upper 2010 and lower 2020 support surfaces.

FIG. 20B shows additional examples of flexure prongs 2030A, 2030B, 2030C having multiple variations in the number of bends or curves. Adding larger bends, double-bends, or changes in the cross-section could be used to increase or decrease the bending stiffness, by increasing or decreasing the moment arm, distance from the bending axis, or moment of inertia (cross-sectional area). More bends also provide more length and surface area for deformation of the flexure to extend or bend further. This could also show how the flexures could be printed independently or separately from the support surfaces.

FIG. 20C shows an illustrative example of a flexure prong 2030A which splits into two or more branches. Splitting or joining of one or more bends may allow for better distribution of forces to the connecting support surfaces. For example a support surface might itself be open or porous (e.g. a donut shape) with fewer regions available for connection of the flexure prongs.

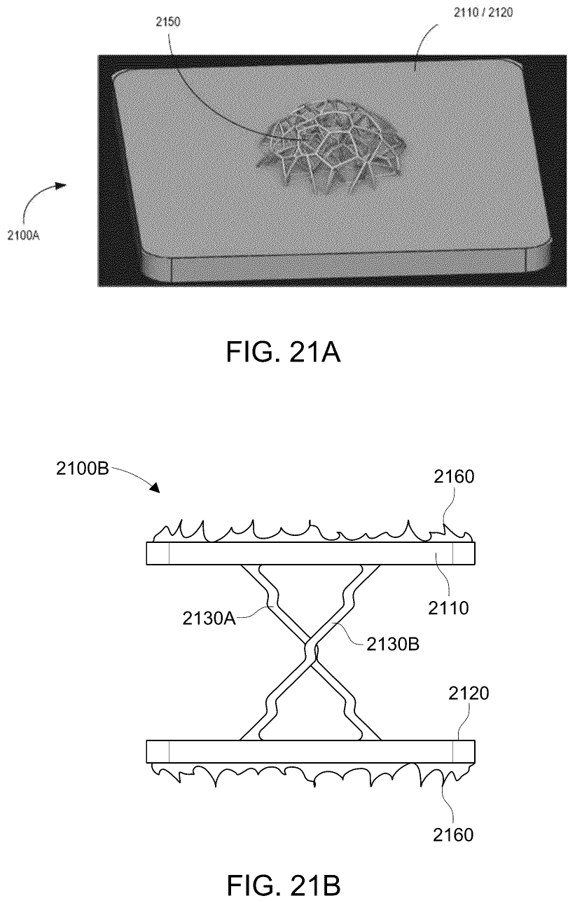

FIG. 21A shows an illustrative example 2100A of a lattice arrangement 2150 on a support surface 2110, 2120 for facilitating a tighter bond between the support surface and additional material which will be placed within the gap between the plates. This feature could also be used as a reference marker or object on imaging to quantify the function or performance of the structure.

FIG. 21B shows an illustrative example of a compliant support mechanism 2100B having rough or uneven surfaces 2160 provided on the support layer 2110, 2120 to provide better coupling with adjacent surfaces.

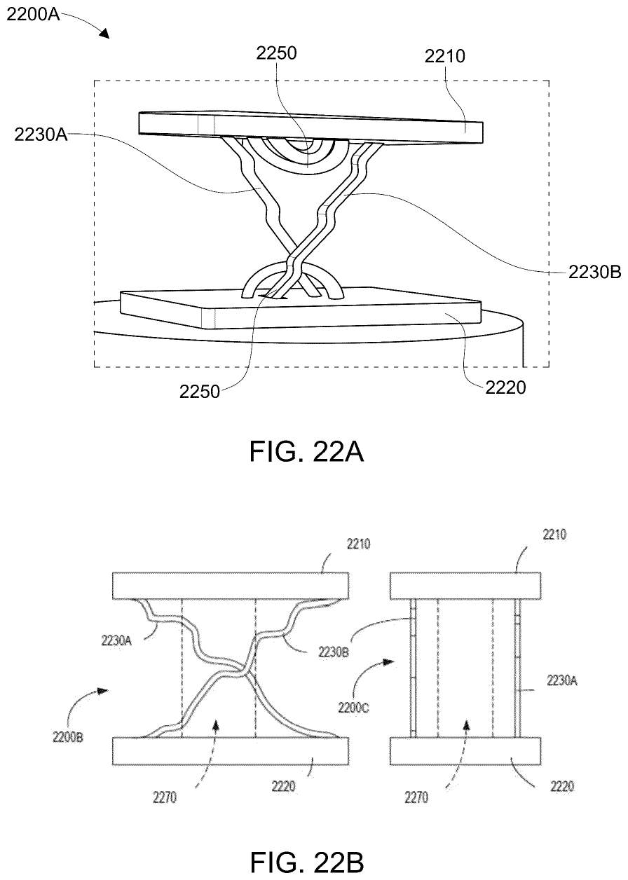

FIG. 22A shows an illustrative example of the lattice arrangement 2250 of FIG. 21A provided on a 3D printed example of a compliant support mechanism 2200A with upper 2210 and lower 2220 support surfaces and flexure prongs 2230A, 2230B.

FIG. 22B shows an illustrative example of a compliant support mechanism 2200B having an open center area 2270 between support layers 2210, 2220 and flexure prongs 2230A,2230B.

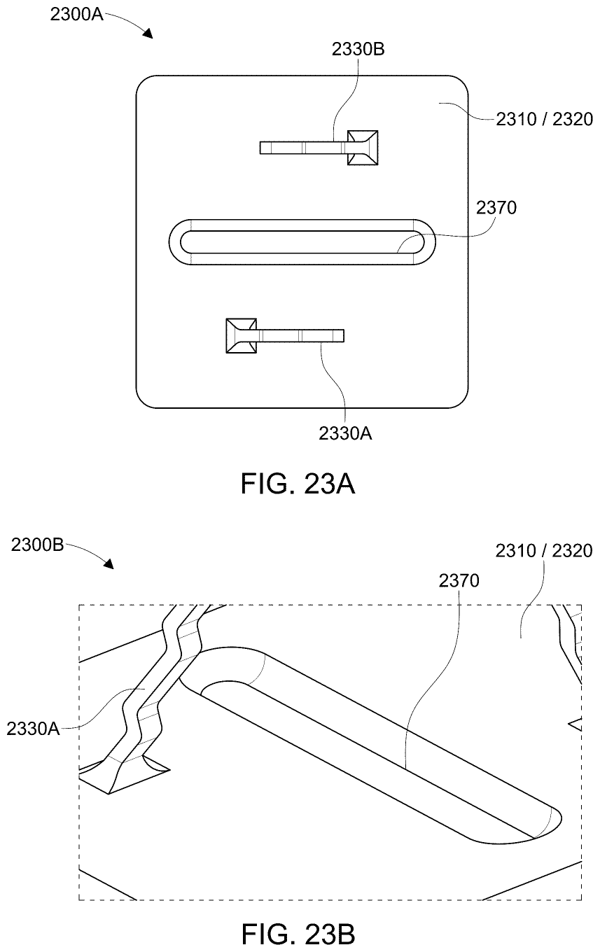

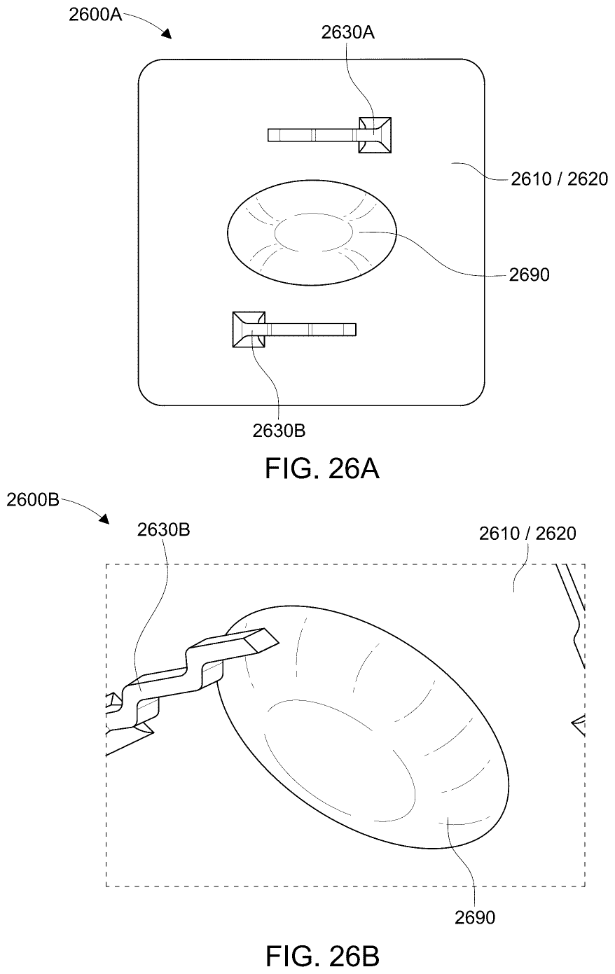

FIG. 23A shows an illustrative example of a support surface 2310, 2320 with flexure prongs 2330A, 2330B having an elongate groove 2370 for guiding or constraining a bearing in accordance with an embodiment. In this embodiment, the compliant support mechanism 2300A could work in tandem with a bearing component, such as a bearing as illustrated in FIG. 26A or FIG. 26B, to provide additional structural support under load and while in motion.

FIG. 23B shows a close up 2300B of the elongate groove 2370 of FIG. 23A, and in this view the elongate groove may allow movement of a bearing along the length of the groove. This may allow translational motion of the bearing relative to the support surface while the bearing provides support for a load placed on the bearing in a perpendicular direction relative to the support surface. A corresponding elongate groove which mirrors the elongate groove of FIG. 23A could similarly constrain the bearing from above.

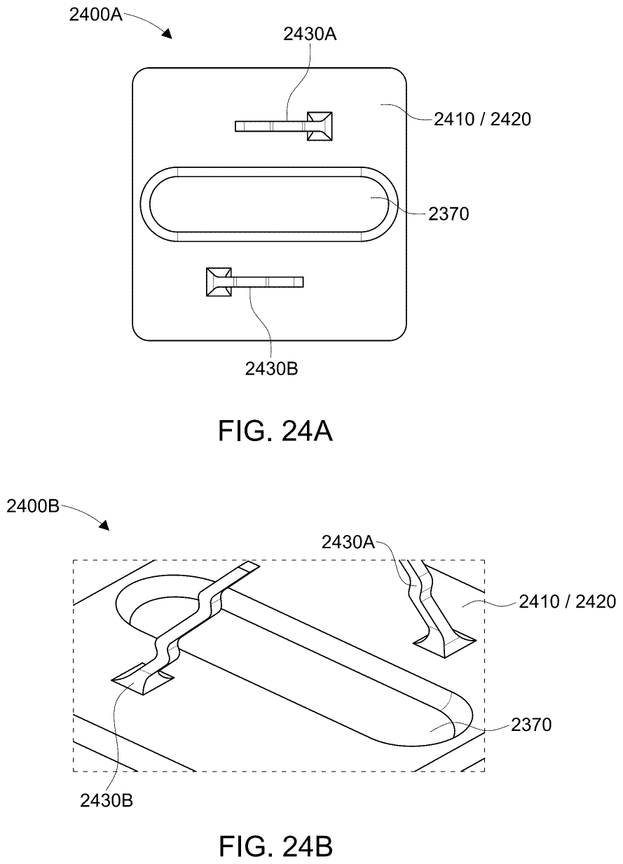

Now referring to FIG. 24A, shown is an illustrative example 2400A of a support surface 2410, 2420 with flexure prongs 2430A, 2430B having an oval groove 2370 for guiding or constraining a bearing in accordance with an embodiment.

FIG. 24B shows a close up 2400B of the oval groove 2370 of FIG. 24A. Similar to the elongate groove shown in FIG. 23A and FIG. 23B, this oval groove 2370 of FIG. 24A and FIG. 24B can constrain a bearing having a larger diameter, along the length of the groove. While the bearing provides support in a perpendicular direction relative to the support surfaces, the arrangement can still provide other types of motion as permitted by the design, such as flexion, extension, translation, or twisting.

While illustrative shapes have been shown and described above, it will be appreciated that the features formed in or on the support surfaces to constrain or guide a support bearing could be either a positive feature or a negative feature (e.g. grooves, concave/convex guides, notches, channels, etc.). The bearings would provide support in a perpendicular or normal direction while also providing compliant movement in translational, rotational, and/or any other types of movements made possible by the location and design of the flexure prongs.

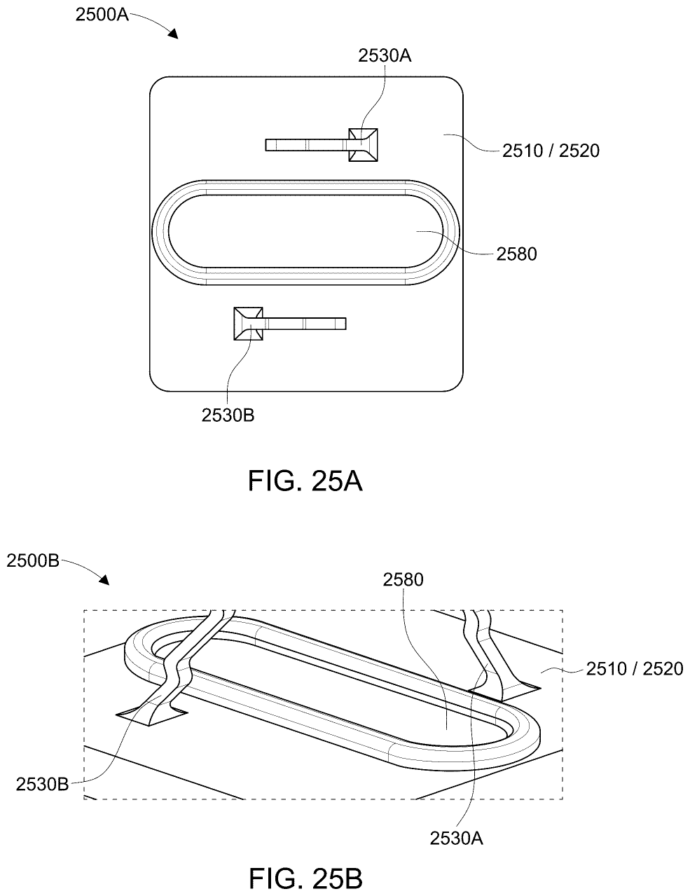

FIG. 25A shows an illustrative example 2500A of a support surface 2510, 2520 with flexure prongs 2530A, 2530B having a raised oval fence 2580 for guiding or constraining a bearing in accordance with an embodiment.

FIG. 25B shows a close up 2500B of the oval fence 2580 of FIG. 25A. In this case, rather than being a depression, there is an oval fence which constrains or guides a bearing within the fence, which allows translational motion along its length. Thus, the guide need not be formed into the support surface, but could instead be formed through additive manufacturing, for example, to the support surface. The guide fence could be made of any number of materials, including metal, ceramic, polymers (e.g. UHMWPE, PEEK) or hybrid combinations of materials.

FIG. 26A shows at 2600A a support surface 2610, 2620 with flexure prongs 2630A, 2630B having an egg shaped concave depression 2690 for constraining the egg shaped bearing in accordance with an embodiment.

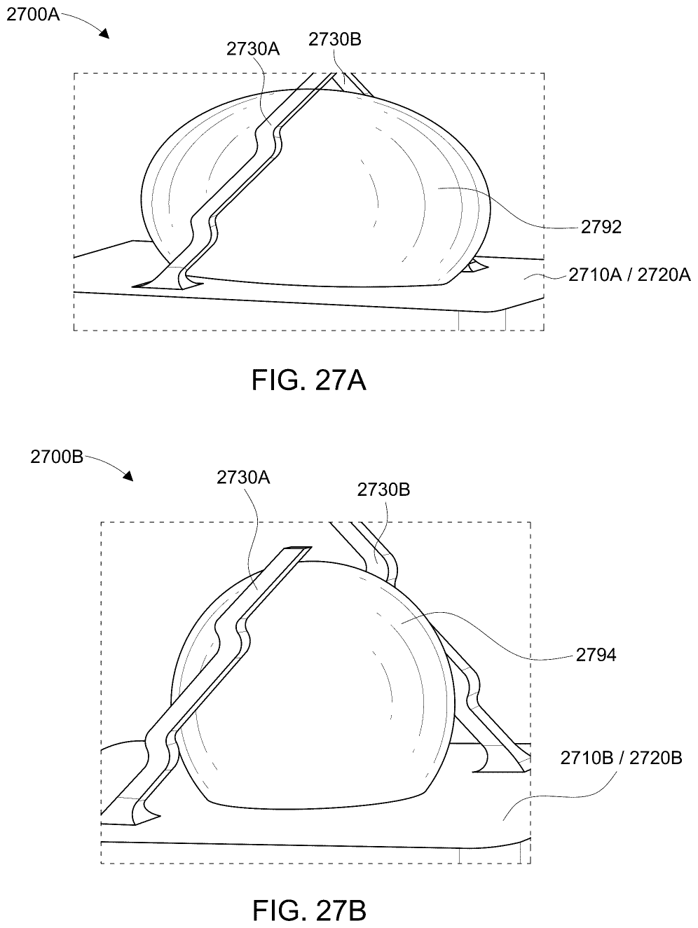

FIG. 26B shows a close up view 2600B of the egg shaped concave depression 2690 of FIG. 27A. As noted above, this concave depression may constrain or guide a bearing in one position, while allowing for some movement as permitted by the arrangement of flexure prongs, such as flexion, extension, translation, or twisting.

FIG. 27A shows at 2700A an egg shaped bearing 2792 being constrained within a correspondingly shaped concave depression in a support plate 2710A, 2720A with flexure prongs 2730A, 2730B in accordance with an embodiment. The bearing could be made of materials such as metal, ceramic, polymers (e.g. UHMWPE, PEEK) or hybrid combinations of materials. The polymers may include elastomers, hydrogels or other biomaterials characterized by their elastic and viscoelastic properties, and may be used in conjunction with the flexural components to provide additional load bearing capacity and a desired flexural profile.

FIG. 27B shows at 2700B a spherical bearing 2794 being constrained within a correspondingly shaped concave depression in a support plate 2710B, 27120B with flexure prongs 2730A, 2730B in accordance with an embodiment. Thus, the bearing 2794 may be fixed in place, or the bearing may be allowed to move along a groove to permit translational motion, and other forms of motion permitted by the design.

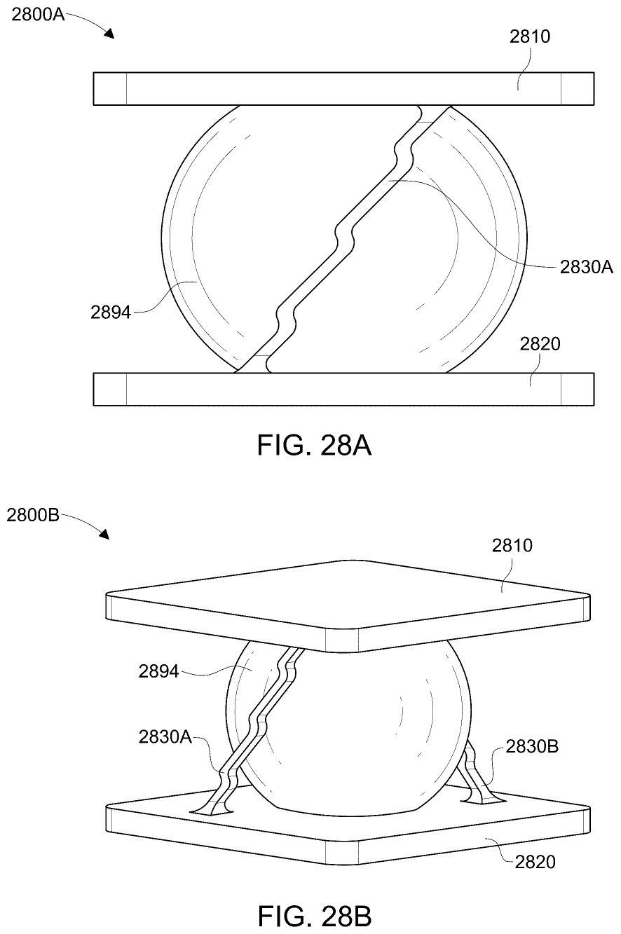

FIG. 28A shows at 2800A a spherical bearing 2894 being constrained between an upper support surface 2810 and a lower support surface 2820 with a flexure prong 2830A in accordance with an embodiment.

FIG. 28B shows at 2800B another view of a spherical bearing 2894 being constrained between an upper support surface 2810 and a lower support surface 2820 with flexure prongs 2830A, 2830B in accordance with an embodiment. It will be appreciated that even as the spherical bearing maintains a gap between the upper and lower support surfaces, the upper and lower support surfaces can still move relative to each other as permitted by the rounded shape of the spherical bearing, and the placement and configuration of the flexure prongs 2830A, 2830B.

It will be appreciated that while the bearing may be built to withstand significant wear and tear, should the bearing wear down, it could be replaced while the complaint support mechanism otherwise remains in situ within a patient.

Thus, in summary, the compliant support mechanism may contain multiple layers that are stacked on top of each other, where each layer of the device may have different flexure elements, different materials, flexure elements characterized by different non-linear or linear paths, or flexure elements bending and curving in different dimensions, in order to provide each layer of the device with its own unique compliance profile, that in turn provides the device as a whole with its own unique compliance profile.

The flexures may have multiple bends in them at specific lengths or varying lengths along the flexure. The number of bends and location of those bends is subject to change depending on the compliance profile required for a particular application. The flexures may also consist of different materials or fibers that are stacked, woven, or laminated together. These materials may follow a single flexure path or one material may dominate while a secondary material fills in engineered defects as described above. However, a common feature is a piecewise, non-linear, multi-bend, kinked shape that generates the compliance that allows for a greater range of motion for flexion, extension, translation, or twisting.

As a specific illustrative example, and not by way of limitation, in an embodiment, the flexure prongs may be square or rectangular, but circular cross-section flexures could also be used.

As previously mentioned, the compliant support mechanism is set up to be additively manufactured such that it may be printed using a 3D printer, for example. Certain design features may better accommodate 3D printing, such as the flexures making a 45 degree angle with horizontal in order to prevent overhang from occurring during the manufacturing process.

While a single material type may be used, in an embodiment, the compliant support mechanism may be printed using two or more different types of materials, which may provide different compliance characteristics depending on which materials are used to print which portion. In some cases, the flexure prongs may be printed in layers from two or materials, giving them certain bending or resiliency characteristics they might not otherwise have if only a single material type is used.

By way of example and not by way of limitation, the compliant support mechanism can be made of different materials including ferrous and non-ferrous metals, polymers, or other biomaterials. For a preferred embodiment for use in orthopaedic implants, biocompatible materials such as titanium or stainless steel would be suitable.

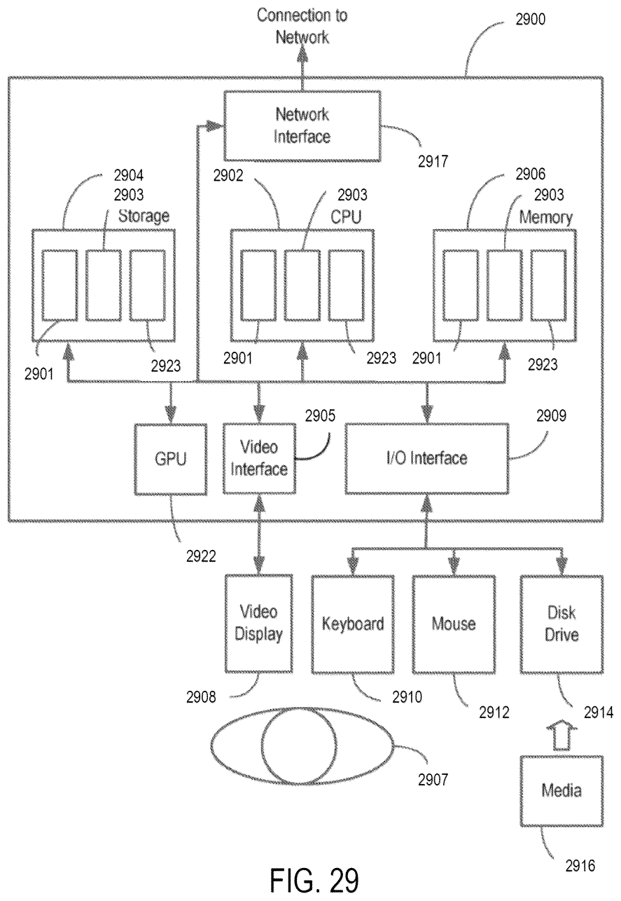

Now referring to FIG. 29 shown is a schematic block diagram of a generic computing device 2900 that may provide a suitable operating environment in one or more embodiments. A suitably configured computer device, and associated communications networks, devices, software and firmware may provide a platform for enabling one or more embodiments as described above. By way of example, FIG. 29 shows a generic computer device 1000 that may include a central processing unit (“CPU”) 2902 connected to a storage unit 2904 and to a random access memory 2906. CPU 2902 may process operating system 2901, application program 2903, and data 2923. The operating system 2901, application program 2903, and data 2923 may be stored in storage unit 2904 and loaded into memory 2906, as may be required. Computer device 2900 may further include a graphics processing unit (GPU) 2922 which is operatively connected to CPU 2902 and to memory 2906 to offload intensive calculations (including, but not limited to image processing or inference or training of deep learning AI models) from CPU 2902 and run these calculations in parallel with CPU 2902. An operator 2910 may interact with the computer device 2900 using a video display 2908 connected by a video interface 2905, and various input/output devices such as a keyboard 2910, pointer 2912, and storage 2914 connected by an I/O interface 2909. In known manner, the pointer 2912 may be configured to control movement of a cursor or pointer icon in the video display 2908, and to operate various graphical user interface (GUI) controls appearing in the video display 2908. The computer device 2900 may form part of a network via a network interface 2911, allowing the computer device 2900 to communicate with other suitably configured data processing systems or circuits. A non-transitory medium 2916 may be used to store executable code embodying one or more embodiments of the present method on the generic computing device 2900.

In an embodiment, computer-aided design (“CAD”) or computer-aided engineering (“CAE”) tools could be used or developed to produce and analyze the performance of the compliant support mechanism design, or an embodiment of the compliant support mechanism design as a spinal implant for example. These CAD or CAE tools may use a variety of numerical methods, such as finite element modeling, to examine the resulting movements, deformations and stresses in a particular design under varying forces, loads, and boundary conditions. Other computational modeling tools, such as rigid body or multi-body solvers, musculoskeletal models, finite element models and related analytical or numerical methods or AI/DL models, could be used to represent or identify patient characteristics to define the loads and boundary conditions inputs applied to the embodiment of the compliant support mechanism in the form of an implantable medical device, such as a spinal disc replacement.

By way of example, and not by way of limitation, the implant design could subsequently be optimized to meet the required design criteria under these conditions using CAE tools like finite element analysis, for example. CAE tools like finite element analysis (“FEA”) could run a large number of simulations to vary the design parameters to develop a better insight into the high performance design concepts and identify the ones most suitable to mechanical performance criteria like durability, flexibility, strength or stiffness, or as a combination of desired performance criteria to meet the needs of a particular patient or surgeon. The FEA may include, but is not limited to:
Structural Analysis: Evaluation of the mechanical strength and stiffness of the compliant support mechanism or a related embodiment under various loading conditions, simulating how the design will withstand forces without failing or undergoing excessive deformation. Structural analysis may be crucial for ensuring the durability and reliability of the design over time.
Dynamic Analysis: Assessment of the compliant support mechanism's behavior under dynamic loading conditions, such as impact or vibration. This type of analysis is important for designs subjected to cyclic loading, like spinal disc replacements, to ensure they can withstand repetitive stresses without fatigue failure.
Thermal Analysis: Investigation of how temperature variations affect the design, including material expansion, contraction, or any other thermal-induced stresses. Although temperature variations are less of a concern for many implants, thermal analysis may be critical for multi-material designs that are sensitive to temperature changes.
Fluid-Structure Interaction (FSI): Examination of the interaction between the compliant support mechanism and any surrounding fluids, be it biological or otherwise. This may be particularly important for potential applications in cardiovascular implants, such as stents or heart valves, where blood flow dynamics and the device's impact on these dynamics need to be thoroughly understood.
Wear Analysis: Prediction of the wear behavior and the potential generation of wear particles is important in the design and use of the compliant support mechanism, especially for embodiments relating to the use of sliding bearing components or contacting material interfaces in the body, such as for joint replacements. Wear analysis helps in understanding how materials will behave against each other in the body and can inform material selection and design choices to minimize wear.
Multiphysics Analysis: Simultaneous consideration of multiple physical phenomena, such as mechanical, thermal, and electrical effects. Multiphysics analysis integrates these different analyses to provide a comprehensive understanding of the compliant support mechanism's behavior in the complex physiological environment.

Each type of analysis may play a critical role in the development and validation process of the compliant support mechanism, especially in related embodiments to implantable medical devices, ensuring they meet the highest standards of safety and effectiveness before being implanted in patients. The choice of analysis depends on the specific characteristics and intended use of the implant.

Based on the CAE, implant designs could be then fabricated to confirm, verify or validate the results of the computational models and simulations.

In an embodiment, an artificial intelligence/machine learning engine may be employed to maintain a database of different types of materials used, and different shapes, profiles, and configurations of the flexure prongs employed in order to track long term performance of each type of compliant support mechanism built in accordance with the present invention. This data can provide feedback to the AI/ML engine to develop models to predict how a given configuration will modify or change a compliance profile. By modeling alternative designs before manufacture, a better design can be predicted without wasting resources. Upon collecting enough data points to develop an accurate model, the AI/ML engine may recommend particular flexure prong designs and configurations to support a given use case.

By way of example, and not by way of limitation, various AI, ML, and deep learning models could aid designers in creating tailored compliance support mechanisms or embodiments as patient-specific implants. For example, Convolutional Neural Networks (CNNs) can analyze medical imaging data for a particular patient to understand patient anatomy intricacies, enabling an implant device to be specifically customized, adjusted, or altered for a particular patient. Generative Adversarial Networks (GANs) might also be used to propose new implant designs based on collected data, allowing the device implant designs to be improved over time. Reinforcement learning could be used to optimize the design process by iteratively improving design parameters for better outcomes. In addition, supervised learning algorithms could predict the compatibility of designs with individual patient needs, ensuring high success rates of implant surgeries.

While various illustrative embodiments have been shown and described, it will be appreciated that the scope of the invention will be determined by the following claims.

1. A compliant support mechanism,
comprising: a first support surface;
a second support surface; and
one or more flexure prongs arranged in a configuration therebetween, each flexure prong having a plurality of bends or curves along its length so as to allow the configuration of one or more flexure prongs to be resiliently compliant when subjected to external forces on the support surfaces.

2. The compliant support mechanism of claim 1, wherein the one or more flexure prongs have a compliance profile dependent upon the application.

3. The compliant support mechanism of claim 2, wherein at least one of the one or more flexure prongs have a varying cross section across its length.

4. The compliant support mechanism of claim 2, wherein at least one of the one or more flexure prongs have deliberately introduced external notches, engineered defects, or internal pores along its length to facilitate the compliance profile.

5. The compliant support mechanism of claim 1, wherein at least one of the one or more flexure prongs splits into two or more branches.

6. The compliant support mechanism of claim 1, wherein the configuration of the plurality of flexure prongs allows the complaint support mechanism to be printed as a monolithic structure.

7. The compliant support mechanism of claim 1, wherein the support surfaces include one or more features to allow an end of each flexure prong to be attached or coupled to the first or second support surface.

8. The compliant support mechanism of claim 1, further comprising one or more additional surface layers and an arrangement of a plurality of flexure prongs between the additional surface layers and the first or second support surface.

9. The compliant support mechanism of claim 8, further comprising an aperture or hole provided in one of the support surfaces to allow a flexure prong to pass through the aperture or hole.

10. The compliant support mechanism of claim 1, wherein the first support surface or the second support surface includes a lattice structure for providing a securing or alignment point.

11. The compliant support mechanism of claim 1, wherein at least one of the first support surface and the second support surface includes a bearing guide for constraining or guiding a bearing positioned between the first and second support surfaces.

12. The compliant support mechanism of claim 11, wherein the bearing guide comprises one or more of a groove, a concave depression, a notch, a channel, or a raised fence adapted to constrain a bearing positioned between the first and second support surfaces.

13. The compliant support mechanism of claim 12, further comprising a bearing which provides support in a perpendicular direction relative to at least one of the first support
surface and the second support surface, while permitting a range of motion including one or more of flexion, extension, translation, and twisting.

14. The compliant support mechanism of claim 12, wherein the bearing is replaceable while the compliant support mechanism remains in situ within a patient.

15. A method of manufacturing the compliant support mechanism of claim 1 for a particular patient, comprising:
analyzing patient attributes, medical history and medical imaging data for the particular patient to understand patient anatomical intricacies specific to the patient; and
3D printing the compliant support mechanism based on the anatomical intricacies specific to the patient.

16. The method of claim 15, further comprising 3D printing the first support surface, the one or more flexure prongs, and the second support surface as a monolithic structure.

17. A method of customizing the compliant support mechanism of claim 1 for a particular patient, comprising:
analyzing patient attributes, medical history and medical imaging data for the particular patient to understand patient anatomy intricacies specific to the patient; and
adjusting the compliant support mechanism in view of the patient anatomy intricacies specific to the patient.

18. The method of claim 17, further comprising employing one or more of analytical methods, numerical methods, computational models, and artificial intelligence/machine learning methods to generate and maintain a database of different types of materials used, and different shapes, profiles, and configurations of the flexure prongs employed in order to design and optimize the long term performance of each type of compliant support mechanism.

19. The method of claim 18, further comprising providing feedback to the AI/ML engine to train and develop models to predict how a given device configuration will modify or change a compliance profile.
